(19) United States
(12) Patent Application Publication
(10) Pub. No.: US 2004/0235108 A1
(43) Pub. Date: Nov. 25, 2004

(54) Monoclonal antibodies that specifically bind a tumor antigen

(76) Inventors: Luigi Grasso, Bala Cynwyd, PA (US); Nicholas C. Nicolaides, Boothwyn, PA (US); Philip M. Sass, Audubon, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/851,786

(22) Filed: May 21, 2004

Related U.S. Application Data
(60) Provisional application No. 60/472,940, filed on May 23, 2003.

(51) Int. Cl. C07H 21/04; A61K 39/395; C07K 16/30; C12N 5/06

(52) U.S. Cl. 435/69.1; 530/388.22; 435/334; 435/320.1; 536/23.53

(57) ABSTRACT

Monoclonal antibodies that specifically bind to the tetrameric form of the alpha-folate receptor and not the monomeric form are provided. The antibodies are useful in the treatment of certain cancers, particularly cancers that have increased cell surface expression of the alpha-folate receptor (“FR-α”), such as ovarian cancer. Hybridoma cells expressing the monoclonal antibodies, antibody derivatives, such as chimeric and humanized monoclonal antibodies, antibody fragments, mammalian cells expressing the monoclonal antibodies, derivatives and fragments, and methods of detecting and treating cancer using the antibodies, derivatives and fragments also are provided.

### CROSS-REFERENCE TO RELATED APPLICATIONS

This application claims the benefit of U.S. Provisional Application No. 60/472,940, filed May 23, 2003, the contents of which are incorporated herein by reference.

### FIELD OF THE INVENTION

This invention relates to novel monoclonal antibodies that specifically bind to the tetrameric form of the alpha-folate receptor and not the monomeric form. The antibodies are useful in the treatment of certain cancers, particularly cancers that have increased cell surface expression of the alpha-folate receptor (“FR-α”), such as ovarian cancer. The invention also related to hybridoma cells expressing the monoclonal antibodies, antibody derivatives, such as chimeric and humanized monoclonal antibodies, antibody fragments, mammalian cells expressing the monoclonal antibodies, derivatives and fragments, and methods of detecting and treating cancer using the antibodies, derivatives and fragments.

### BACKGROUND OF THE INVENTION

There are two major isoforms of the human membrane folate binding proteins, α and β. The two isoforms have about 70% amino acid sequence homology and differ dramatically in their stereospecificity for some folates. Both isoforms are expressed in both fetal and adult tissue, although normal tissue generally expresses low to moderate amounts of FR-β. FR-α, however, is expressed in normal epithelial cells, and is frequently strikingly elevated in a variety of carcinomas (Ross et al. (1994) Cancer 73(9):2432-2443; Rettig et al. (1988) Proc. Natl. Acad. Sci. USA 85:3110-3114; Campbell et al. (1991) Cancer Res. 51:5329-5338; Coney et al (1991) Cancer Res. 51:6125-6132; Weitman et al. (1992) Cancer Res. 52:3396-3401; Garin-Chesa et al. (1993) Am. J. Pathol. 142:557-567; Holm et al. (1994) APMIS 102:413-419; Franklin et al. (1994) Int. J. Cancer 8 (Suppl.):89-95. Miotti et al. (1987) Int. J. Cancer 39:297-303; and Vegglan et al. (1989) Tumori 75:510-513). FR-α is overexpressed in greater than 90% of ovarian carcinomas (Sudimack and Lee (2000)Adv. Drug Deliv. Rev. 41(2):147-62).

Administration of antibodies against the folate binding protein has been proposed as a strategy for treatment of ovarian cancer.

In 1987, Miotti et al. described three new monoclonal antibodies that recognized antigens on human ovarian carcinoma cells (Miotti et al. (1987) Int. J. Cancer 39(3):297-303). One of these was designated MOv18, which recognizes a 38 kDa protein on the surface of choriocarcinoma cells. MOv18 is a murine, IgG1, kappa antibody and mediates specific cell lysis of the ovarian carcinoma cell line, IGROV1. Alberti et al. ((1990) Biochem. Biophys. Res. Commun. 171(3):1051-1055) showed that the antigen recognized by MOv18 was a GPI-linked protein. This was subsequently identified as the human folate binding protein (Coney et al. (1991) Cancer Res. 51(22):6125-6132). Tomassetti et al showed that MOv18 recognizes a soluble form and a GPI-anchored form of the folate binding protein in IGROV1 cells (Tomassetti et al. (1993) FEBS Lett. 317(1-2):143-146). Subsequent work combined the variable regions of the mouse MOv 18 with human IgG 1 (kappa) constant region to create a chimerized MOv 18 antibody. The chimerized antibody mediated higher and more specific lysis of IGROV1 cells at 10-100 fold lower antibody concentrations (Coney et al. (1994) Cancer Res. 54(9):2448-2455). The 38 kDa antigen appears to be the monomeric form of FR-α.

U.S. Pat. No. 5,952,484 describes a humanized antibody that binds to a 38 kDa protein (FR-α). The antibody was named LK26, after the antigen by the same name. The original mouse monoclonal antibody was described by Rettig in European Patent Application No. 86104170.5 (published as EP0197435 and issued in the U.S. as U.S. Pat. No. 4,851,332).

Ovarian cancer is the major cause of death due to gynecological malignancy. Although chemotherapy is the recommended treatment and has enjoyed some success, the 5-year survival term is still less than 40%.

A difficult problem in antibody therapy in cancer is that often the target of the antibody is expressed by normal tissues as well as cancerous tissues. Thus, the antibodies that are used to kill cancer cells also have a deleterious effect on normal cells. Finding unique targets or targets that are preferentially expressed in cancer tissues has proven difficult in many cancers. More effective antibody therapies for ovarian and other FR-α bearing cancers that avoids the problem of reactivity with normal tissues are needed.

### SUMMARY OF THE INVENTION

It has been discovered that tumors that overexpress FR-α tend to favor the formation of tetrameric forms of FR-α. Without wishing to be bound by any particular theory, it is believed that the formation of the tetrameric form of FR-α is driven by a mass effect due to the accumulation of larger amounts of FR-α on the surface of tumor cells. Previously, other researchers only found higher molecular weight species of FR-α in gel filtration assays which represented FR-α inserted into Triton X-100 micelles via their hydrophobic tails (Holm et al. (1997) Biosci. Reports 17(4):415-427). Tetrameric forms of FR-α on the surface of tumors has not been described previously.

The invention provides antibodies that specifically binds to the tetrameric form of FR-α and not the monomeric form wherein the antibody is distinguished from mAb LK26 in that (a) the antibody binds to an epitope other than the epitope of mAb LK26; (b) the antibody binds with greater affinity than mAb LK26; or (c) the antibody out-competes mAb LK26 for binding to the tetrameric form of FR-α.

The antibody of the invention has an affinity of at least about 1×10⁻⁷M, 1×10⁻⁸M, 1×10⁻⁹M, 1×10⁻¹⁰M, 1×10⁻¹¹M, or 1×10⁻¹²M, and recognizes a disulfide-dependent epitope.

The antibody of the invention may be a chimeric antibody, including, but not limited to a human-mouse chimeric antibody. The antibody of the invention may also be a humanized antibody. The invention also provides: hybridoma cells that express the antibodies of the invention; polynucleotides that encode the antibodies of the invention; vectors comprising the polynucleotides that encode the antibodies of the invention; and expression cells comprising the vectors of the invention.

The invention also provides a method of producing an antibody that specifically binds to the tetrameric form of FR-α and not the monomeric form wherein the antibody is distinguished from mAb LK26 in that (a) the antibody binds to an epitope other than the epitope of mAb LK26; (b) the antibody binds with greater affinity than mAb LK26; or (c) the antibody out-competes mAb LK26 for binding to said tetrameric form of FR-α. The method comprising the step of culturing the hybridoma cell that expresses an antibody of the invention or an expression cell that comprises a vector containing a polynucleotide encoding an antibody of the invention. The expression cells of the invention may be insect cells, and animal cells, preferably, mammalian cells.

The invention further provides a method of inhibiting the growth of dysplastic cells associated with increased expression of FR-α comprising administering to a patient with such dysplastic cells a composition comprising an antibody that specifically binds to the tetrameric form of FR-α wherein said antibody is distinguished from LK26 in that (a) the antibody binds to an epitope other than the epitope of LK26, (b) the antibody binds with greater affinity than LK26; or (c) the antibody out-competes mAb LK26 for binding to said tetrameric form of FR-α. The method may be used for various dysplastic conditions, such as, but not limited to ovarian cancer. In preferred embodiments, the patients are human patients. In some embodiments, the antibodies are conjugated to immunotoxic agents such as, but not limited to radionuclides, toxins, and chemotherapeutic agents.

### BRIEF DESCRIPTION OF THE DRAWINGS

FIG. 1 shows a western blot of tumor cells showing the tetrameric and monomeric forms of FR-α.

FIG. 2 shows a western blot of Escherichia coli expressed FR-α.

FIG. 3 shows a western blot of FR-α solubilized in the presence or absence of Triton X-100.

### DETAILED DESCRIPTION OF ILLUSTRATIVE EMBODIMENTS

The reference works, patents, patent applications, and scientific literature, including accession numbers to GenBank database sequences that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Standard reference works setting forth the general principles of recombinant DNA technology known to those of skill in the art include Ausubel et al., CURRENT PROTOCOLS IN MOLECULAR BIOLOGY, John Wiley & Sons, New York (1998); Sambrook et al. MOLECULAR CLONING: A LABORATORY MANUAL,2D ED., Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991).

The invention provides a method for decreasing the growth of cancer cells and the progression of neoplastic disease using monoclonal antibodies that specifically bind to the tetrameric form of the mammalian FR-α. The method of the invention may be used to modulate the growth of cancer cells and the progression of cancer in mammals, including humans. The cancer cells that may be inhibited include all cancer cells that have an increased expression of FR-α in relation to normal human tissues, particularly ovarian cancer cells.

Without wishing to be bound by any particular theory of operation, it is believed that the increased expression of FR-α in cancer cells results in an increased association of monomeric form of FR-α to form tetrameric forms of FR-α on the surface of the cells. Therefore, cancer cells have an increased expression of tetrameric forms of FR-α relative to normal tissues. Thus, the tetrameric form of FR-α is an ideal target for antibody therapy in cancer.

As used herein, the term “epitope” refers to the portion of an antigen to which a monoclonal antibody specifically binds.

As used herein, the term “conformational epitope” refers to a discontinuous epitope formed by a spatial relationship between amino acids of an antigen other than an unbroken series of amino acids.

As used herein, the term “tetrameric” refers to a grouping of four identical, or nearly identical units.

As used herein, the term “monomeric” refers to a single unit of a mature protein that assembles in groups with other units.

As used herein, the term “inhibition of growth of dysplastic cells in vitro” means a decrease in the number of tumor cells, in culture, by about 5%, preferably 10%, more preferably 20%, more preferably 30%, more preferably 40%, more preferably 50%, more preferably 60%, more preferably 70%, more preferably 80%, more preferably 90%, and most preferably 100%. In vitro inhibition of tumor cell growth may be measured by assays known in the art, such as the GEO cell soft agar assay.

As used herein, the term “inhibition of growth of dysplastic cells in vivo” means a decrease in the number of tumor cells, in an animal, by about 5%, preferably 10%, more preferably 20%, more preferably 30%, more preferably 40%, more preferably 50%, more preferably 60%, more preferably 70%, more preferably 80%, more preferably 90%, and most preferably 100%. In vivo modulation of tumor cell growth may be measured by assays known in the art.

As used herein, “dysplastic cells” refer to cells that exhibit abnormal growth. Dysplastic cells include, but are not limited to tumors, hyperplasia, and the like.

The term “preventing” refers to decreasing the probability that an organism contracts or develops an abnormal condition.

The term “treating” refers to having a therapeutic effect and at least partially alleviating or abrogating an abnormal condition in the organism. Treating includes maintenance of inhibited tumor growth, and induction of remission.

The term “therapeutic effect” refers to the inhibition of an abnormal condition. A therapeutic effect relieves to some extent one or more of the symptoms of the abnormal condition. In reference to the treatment of abnormal conditions, a therapeutic effect can refer to one or more of the following: (a) an increase or decrease in the proliferation, growth, and/or differentiation of cells; (b) inhibition (i.e., slowing or stopping) of growth of tumor cells in vivo (c) promotion of cell death; (d) inhibition of degeneration; (e) relieving to some extent one or more of the symptoms associated with the abnormal condition; and (f) enhancing the function of a population of cells. The monoclonal antibodies and derivatives thereof described herein effectuate the therapeutic effect alone or in combination with conjugates or additional components of the compositions of the invention.

As used herein, the term “inhibits the progression of cancer” refers to an activity of a treatment that slows the modulation of neoplastic disease toward end-stage cancer in relation to the modulation toward end-stage disease of untreated cancer cells.

As used herein, the term “about” refers to an approximation of a stated value within an acceptable range. Preferably the range is +/−5% of the stated value.

As used herein, the term “neoplastic disease” refers to a condition marked by abnormal proliferation of cells of a tissue.

Antibodies

The antibodies of the invention specifically bind the tetrameric form of FR-α and not the monomeric form of FR-α. In some embodiments, the antibodies bind to the same epitope as LK26. In other embodiments, the antibodies bind to an epitope other than that bound by LK26.

Preferred antibodies, and antibodies suitable for use in the method of the invention, include, for example, fully human antibodies, human antibody homologs, humanized antibody homologs, chimeric antibody homologs; Fab, Fab′, F(ab′)₂ and F(v) antibody fragments, single chain antibodies, and monomers or dimers of antibody heavy or light chains or mixtures thereof.

The antibodies of the invention may include intact immunoglobulins of any isotype including types IgA, IgG, IgE, IgD, IgM (as well as subtypes thereof). The light chains of the immunoglobulin may be kappa or lambda.

The antibodies of the invention include portions of intact antibodies that retain antigen-binding specificity, for example, Fab fragments, Fab′ fragments, F(ab′)₂ fragments, F(v) fragments, heavy chain monomers or dimers, light chain monomers or dimers, dimers consisting of one heavy and one light chain, and the like. Thus, antigen binding fragments, as well as full length dimeric or trimeric polypeptides derived from the above-described antibodies are themselves useful.

The expression cells of the invention include any insect expression cell line known, such as for example, Spodoptera frugiperda cells. The expression cell lines may also be yeast cell lines, such as, for example, Saccharomyces cerevisiae and Schizosaccharomyces pombe cells. The expression cells may also be mammalian cells such as, for example Chinese Hamster Ovary, baby hamster kidney cells, human embryonic kidney line 293, normal dog kidney cell lines, normal cat kidney cell lines, monkey kidney cells, African green monkey kidney cells, COS cells, and non-tumorigenic mouse myoblast G8 cells, fibroblast cell lines, myeloma cell lines, mouse NIH/3T3 cells, LMTK31 cells, mouse sertoli cells, human cervical carcinoma cells, buffalo rat liver cells, human lung cells, human liver cells, mouse mammary tumor cells, TR1 cells, MRC 5 cells, and FS4 cells.

A “chimeric antibody” is an antibody produced by recombinant DNA technology in which all or part of the hinge and constant regions of an immunoglobulin light chain, heavy chain, or both, have been substituted for the corresponding regions from another animal's immunoglobulin light chain or heavy chain. In this way, the antigen-binding portion of the parent monoclonal antibody is grafted onto the backbone of another species' antibody. One approach, described in EP 0239400 to Winter et al. describes the substitution one species complementarity determining regions (CDRs) for those of another species, such as substituting the CDRs from human heavy and light chain immunoglobulin variable region domains with CDRs from mouse variable region domains. These altered antibodies may subsequently be combined with human immunoglobulin constant regions to form antibodies that are human except for the substituted murine CDRs which are specific for the antigen. Methods for grafting CDR regions of antibodies may be found, for example in Riechmann et al. (1988) Nature 332:323-327 and Verhoeyen et al. (1988) Science 239:1534-1536.

Chimeric antibodies were thought to circumvent the problem of eliciting an immune response in humans than chimeric antibodies contain less murine amino acid sequences. It was found that the direct use of rodent MAbs as human therapeutic agents led to human anti-rodent antibody (“HARA”) responses which occurred in a significant number of patients treated with the rodent-derived antibody (Khazaeli, et al. (1994) Immunother. 15:42-52).

As a non-limiting example, a method of performing CDR grafting may be performed by sequencing the mouse heavy and light chains of the antibody of interest that binds to the target antigen (e.g., FR-α) and genetically engineering the CDR DNA sequences and imposing these amino acid sequences to corresponding human V regions by site directed mutagenesis. Human constant region gene segments of the desired isotype are added, and the “humanized” heavy and light chain genes are co-expressed in mammalian cells to produce soluble humanized antibody. A typical expression cell is a Chinese Hamster Ovary (CHO) cell. Suitable methods for creating the chimeric antibodies may be found, for example, in Jones et al. (1986) Nature 321:522-525; Riechmann (1988) Nature 332:323-327; Queen et al. (1989) Proc. Nat. Acad. Sci. USA 86:10029; and Orlandi et al. (1989) Proc. Natl. Acad. Sci. USA 86:3833.

Further refinement of antibodies to avoid the problem of HARA responses led to the development of “humanized antibodies.” Humanized antibodies are produced by recombinant DNA technology, in which at least one of the amino acids of a human immunoglobulin light or heavy chain that is not required for antigen binding has been substituted for the corresponding amino acid from a nonhuman mammalian immunoglobulin light or heavy chain. For example, if the immunoglobulin is a mouse monoclonal antibody, at least one amino acid that is not required for antigen binding is substituted using the amino acid that is present on a corresponding human antibody in that position. Without wishing to be bound by any particular theory of operation, it is believed that the “humanization” of the monoclonal antibody inhibits human immunological reactivity against the foreign immunoglobulin molecule.

Queen et al. (1989) Proc. Nat. Acad. Sci. USA 86:10029-10033 and WO 90/07861 describe the preparation of a humanized antibody. Human and mouse variable framework regions were chosen for optimal protein sequence homology. The tertiary structure of the murine variable region was computer-modeled and superimposed on the homologous human framework to show optimal interaction of amino acid residues with the mouse CDRs. This led to the development of antibodies with improved binding affinity for antigen (which is typically decreased upon making CDR-grafted chimeric antibodies). Alternative approaches to making humanized antibodies are known in the art and are described, for example, in Tempest (1991) Biotechnology 9:266-271.

“Single chain antibodies” refer to antibodies formed by recombinant DNA techniques in which immunoglobulin heavy and light chain fragments are linked to the Fᵥregion via an engineered span of amino acids. Various methods of generating single chain antibodies are known, including those described in U.S. Pat. No. 4,694,778; Bird (1988) Science242:423-442; Huston et al. (1988) Proc. Natl. Acad. Sci. USA 85:5879-5883; Ward et al. (1989) Nature 334:54454; Skerra et al. (1988) Science 242:1038-1041.

The antibodies of the invention may be used alone or as immunoconjugates with a cytotoxic agent. In some embodiments, the cytotoxic agent is a radioisotope, including, but not limited to Lead-212, Bismuth-212, Astatine-211, Iodine-131, Scandium-47, Rhenium-186, Rhenium-188, Yttrium-90, Iodine-123, Iodine-125, Bromine-77, Indium-111, and fissionable nuclides such as Boron-10 or an Actinide. In other embodiments, the cytotoxic agent is a well-known toxins and cytotoxic drugs, including but not limited to ricin, modified Pseudomonas enterotoxin A, calicheamicin, adriamycin, 5-fluorouracil, and the like. Conjugation of antibodies and antibody fragments to such cytotoxic agents is well-known in the literature.

The antibodies of the invention include derivatives that are modified, e.g., by the covalent attachment of any type of molecule to the antibody such that covalent attachment does not prevent the antibody from binding to its epitope. Examples of suitable derivatives include, but are not limited to glycosyled antibodies and fragments, acetyled antibodies and fragments, pegylated antibodies and fragments, phosphylated antibodies and fragments, and amidated antibodies and fragments. The antibodies and derivatives thereof of the invention may themselves by derivatized by known protecting/blocking groups, proteolytic cleavage, linkage to a cellular ligand or other proteins, and the like. Further, the antibodies and derivatives thereof of the invention may contain one or more non-classical amino acids.

The invention also encompasses fully human antibodies such as those derived from peripheral blood mononuclear cells of ovarian cancer patients. Such cells may be fused with myeloma cells, for example to form hybridoma cells producing fully human antibodies against FR-α.

Without wishing to be bound by any particular theory of operation, it is believed that the antibodies of the invention are particularly useful to bind the tetrameric form of FR-α due to an increased avidity of the antibody as both “arms” of the antibody (F_{ab} fragments) bind to separate FR-α molecules that make up the tetramer. This leads to a decrease in the dissociation (K_{d}) of the antibody and an overall increase in the observed affinity (K_{D}). This is an especially good feature for targeting tumors as the antibodies of the invention will bind more tightly to tumor tissue than normal tissue.

Methods of Producing Antibodies to FR-α

Immunizing Animals

The invention also provides methods of producing monoclonal antibodies that specifically bind to the tetrameric form of FR-α. Tetrameric FR-α may be purified from cells or from recombinant systems using a variety of well-known techniques for isolating and purifying proteins. For example, but not by way of limitation, tetrameric FR-α may be isolated based on the apparent molecular weight of the protein by running the protein on an SDS-PAGE gel and blotting the proteins onto a membrane. Thereafter, the appropriate size band corresponding to the tetrameric form of FR-α may be cut from the membrane and used as an immunogen in animals directly, or by first extracting or eluting the protein from the membrane. As an alternative example, the protein may be isolated by size-exclusion chromatography alone or in combination with other means of isolation and purification. Other means of purification are available in such standard reference texts as Zola, MONOCLONAL ANTIBODIES: PREPARATION AND USE OF MONOCLONAL ANTIBODIES AND ENGINEERED ANTIBODY DERIVATIVES (BASICS: FROM BACKGROUND TO BENCH) Springer-Verlag Ltd., New York, 2000; BASIC METHODS IN ANTIBODY PRODUCTION AND CHARACTERIZATION, Chapter 11, “Antibody Purification Methods,” Howard and Bethell, Eds., CRC Press, 2000;ANTIBODY ENGINEERING (SPRINGER LAB MANUAL), Kontermann and Dubel, Eds., Springer-Verlag, 2001.

One strategy for generating antibodies against FR-α involves immunizing animals with the tetrameric form of FR-α. Animals so immunized will produce antibodies against the protein. Standard methods are known for creating monoclonal antibodies including, but are not limited to, the hybridoma technique (see Kohler & Milstein (1975)Nature 256:495-497); the trioma technique; the human B-cell hybridoma technique (see Kozbor et al. (1983) Immunol. Today 4:72) and the EBV hybridoma technique to produce human monoclonal antibodies (see Cole, et al. in MONOCLONAL ANTIBODIES AND CANCER THERAPY, Alan R. Liss, Inc., 1985, pp. 77-96).

Screening for Antibody Specificity

Screening for antibodies that specifically bind to the tetrameric form of FR-α may be accomplished using an enzyme-linked immunosorbent assay (ELISA) in which microtiter plates are coated with the tetrameric form of FR-α. Antibodies from positively reacting clones can be further screened for reactivity in an ELISA-based assay to the monomeric form of FR-α using microtiter plates coated with the monomeric form of FR-α. Clones that produce antibodies that are reactive to the monomeric form of FR-α are eliminated, and clones that produce antibodies that are reactive to the tetrameric form only are selected for further expansion and development.

Confirmation of reactivity of the antibodies to the tetrameric form of FR-α may be accomplished, for example, using a Western Blot assay in which protein from ovarian cancer cells and purified tetrameric and monomeric FR-α are run on an SDS-PAGE gel under reducing and non-reducing conditions, and subsequently are blotted onto a membrane. The membrane may then be probed with the putative anti-tetrameric FR-α antibodies. Reactivity with the 152 kDa form of FR-α under non-reducing conditions and not the 38 kDa form of FR-α (under reducing or non-reducing conditions) confirms specificity of reactivity for the tetrameric form of FR-α.

The antibodies and derivatives thereof of the invention have binding affinities that include a dissociation constant (K_{d}) of less than 1×10⁻². In some embodiments, the K_{d} is less than 1×10⁻³. In other embodiments, the K_{d} is less than 1×10⁻⁴. In some embodiments, the K_{d} is less than 1×10⁻⁵. In still other embodiments, the K_{d} is less than 1×10⁻⁶. In other embodiments, the K_{d} is less than 1×10⁻⁷. In other embodiments, the K_{d} is less than 1×10⁻⁸. In other embodiments, the K_{d} is less than 1×10⁻⁹. In other embodiments, the K_{d} is less than 1×10⁻¹⁰. In still other embodiments, the K_{d} is less than 1×10⁻¹¹. In some embodiments, the K_{d} is less than 1×10⁻¹². In other embodiments, the K_{d} is less than 1×10⁻¹³. In other embodiments, the K_{d} is less than 1×10⁻¹⁴. In still other embodiments, the K_{d} is less than 1×10⁻¹⁵.

Production of Antibodies

Antibodies of the invention may be produced in vivo or in vitra For in vivo antibody production, animals are generally immunized with an immunogenic portion of FR-α (preferably tetrameric FR-α). The antigen is generally combined with an adjuvant to promote immunogenicity. Adjuvants vary according to the species used for immunization. Examples of adjuvants include, but are not limited to: Freund's complete adjuvant (“FCA”), Freund's incomplete adjuvant (“FIA”), mineral gels (e.g., aluminum hydroxide), surface active substances (e.g., lysolecithin, pluronic polyols, polyanions), peptides, oil emulsions, keyhole limpet hemocyanin (“KLH”), dinitrophenol (“DNP”), and potentially useful human adjuvants such as Bacille Calmette-Guerin (“BCG”) and corynebacterium parvum. Such adjuvants are also well known in the art.

Immunization may be accomplished using well-known procedures. The dose and immunization regimen will depend on the species of mammal immunized, its immune status, body weight, and/or calculated surface area, etc. Typically, blood serum is sampled from the immunized mammals and assayed for anti-FR-α antibodies using appropriate screening assays as described below, for example.

Splenocytes from immunized animals may be immortalized by fusing the splenocytes (containing the antibody-producing B cells) with an immortal cell line such as a myeloma line. Typically, myeloma cell line is from the same species as the splenocyte donor. In one embodiment, the immortal cell line is sensitive to culture medium containing hypoxanthine, aminopterin, and thymidine (“HAT medium”). In some embodiments, the myeloma cells are negative for Epstein-Barr virus (EBV) infection. In preferred embodiments, the myeloma cells are HAT-sensitive, EBV negative and Ig expression negative. Any suitable myeloma may be used. Murine hybridomas may be generated using mouse myeloma cell lines (e.g, the P3-NS1/1-Ag4-1, P3-x63-Ag8.653 or Sp2/O-Ag14 myeloma lines). These murine myeloma lines are available from the ATCC. These myeloma cells are fused to the donor splenocytes polyethylene glycol (“PEG”), preferably 1500 molecular weight polyethylene glycol (“PEG 1500”). Hybridoma cells resulting from the fusion are selected in HAT medium which kills unfused and unproductively fused myeloma cells. Unfused splenocytes die over a short period of time in culture. In some embodiments, the myeloma cells do not express immunoglobulin genes.

Hybridomas producing a desired antibody which are detected by screening assays such as those described below, may be used to produce antibodies in culture or in animals. For example, the hybridoma cells may be cultured in a nutrient medium under conditions and for a time sufficient to allow the hybridoma cells to secrete the monoclonal antibodies into the culture medium. These techniques and culture media are well known by those skilled in the art. Alternatively, the hybridoma cells may be injected into the peritoneum of an unimmunized animal. The cells proliferate in the peritoneal cavity and secrete the antibody, which accumulates as ascites fluid. The ascites fluid may be withdrawn from the peritoneal cavity with a syringe as a rich source of the monoclonal antibody.

Another non-limiting method for producing human antibodies is described in U.S. Pat. No. 5,789,650 which describes transgenic mammals that produce antibodies of another species (e.g., humans) with their own endogenous immunoglobulin genes being inactivated. The genes for the heterologous antibodies are encoded by human immunoglobulin genes. The transgenes containing the unrearranged immunoglobulin encoding regions are introduced into a non-human animal. The resulting transgenic animals are capable of functionally rearranging the transgenic immunoglobulin sequences and producing a repertoire of antibodies of various isotypes encoded by human immunoglobulin genes. The B-cells from the transgenic animals are subsequently immortalized by any of a variety of methods, including fusion with an immortalizing cell line (e.g., a myeloma cell).

Antibodies against FR-α may also be prepared in vitro using a variety of techniques known in the art. For example, but not by way of limitation, fully human monoclonal antibodies against FR-α may be prepared by using in vitro-primed human splenocytes (Boerner et al. (1991) J. Immunol. 147:86-95).

Alternatively, for example, the antibodies of the invention may be prepared by “repertoire cloning” (Persson et al. (1991) Proc. Nat. Acad. Sci. USA 88:2432-2436; and Huang and Stollar (1991) J. Immunol. Methods 141:227-236). Further, U.S. Pat. No. 5,798,230 describes preparation of human monoclonal antibodies from human B antibody-producing B cells that are immortalized by infection with an Epstein-Barr virus that expresses Epstein-Barr virus nuclear antigen 2 (EBNA2). EBNA2, required for immortalization, is then inactivated resulting in increased antibody titers.

In another embodiment, antibodies against FR-α are formed by in vitro immunization of peripheral blood mononuclear cells (“PBMCs”). This may be accomplished by any means known in the art, such as, for example, using methods described in the literature (Zafiropoulos et al. (1997) J Immunological Methods 200:181-190).

In one embodiment of the invention, the procedure for in vitroimmunization is supplemented with directed evolution of the hybridoma cells in which a dominant negative allele of a mismatch repair gene such as PMS1, PMS2, PMS2-134, PMSR2, PMSR3, MLH1, MLH2, MLH3, MLH4, MLH5, MLH6, PMSL9, MSH1, and MSH2 is introduced into the hybridoma cells after fusion of the splenocytes, or to the myeloma cells before fusion. Cells containing the dominant negative mutant will become hypermutable and accumulate mutations at a higher rate than untransfected control cells. A pool of the mutating cells may be screened for clones that produce higher affinity antibodies, or that produce higher titers of antibodies, or that simply grow faster or better under certain conditions. The technique for generating hypermutable cells using dominant negative alleles of mismatch repair genes is described in U.S. Pat. No. 6,146,894, issued Nov. 14, 2000. Alternatively, mismatch repair may be inhibited using the chemical inhibitors of mismatch repair described by Nicolaides et al. in WO 02/054856 “Chemical Inhibitors of Mismatch Repair” published Jul. 18, 2002. The technique for enhancing antibodies using the dominant negative alleles of mismatch repair genes or chemical inhibitors of mismatch repair may be applied to mammalian expression cells expressing cloned immunoglobulin genes as well. Cells expressing the dominant negative alleles can be “cured” in that the dominant negative allele can be turned off, if inducible, eliminated from the cell, and the like such that the cells become genetically stable once more and no longer accumulate mutations at the abnormally high rate.

Methods of Reducing the Growth of Tumor Cells

The methods of the invention are suitable for use in humans and non-human animals identified as having a neoplastic condition associated with an increased expression of FR-α. Non-human animals which benefit from the invention include pets, exotic (e.g., zoo animals) and domestic livestock. Preferably the non-human animals are mammals.

The invention is suitable for use in a human or animal patient that is identified as having a dysplastic disorder that is marked by increased expression of FR-α in the neoplasm in relation to normal tissues. Once such a patient is identified as in need of treatment for such a condition, the method of the invention may be applied to effect treatment of the condition. Tumors that may be treated include, but are not limited to ovarian tumors, renal tumors, lung tumors, fallopian tube tumors, uterine tumors, and certain leukemia cells.

The antibodies and derivatives thereof for use in the invention may be administered orally in any acceptable dosage form such as capsules, tablets, aqueous suspensions, solutions or the like. The antibodies and derivatives thereof may also be administered parenterally. That is via the following routes of administration: subcutaneous, intravenous, intramuscular, intra-articular, intra-synovial, intrasternal, intranasal, topically, intrathecal, intrahepatic, intralesional, and intracranial injection or infusion techniques. Generally, the antibodies and derivatives will be provided as an intramuscular or intravenous injection.

The antibodies and derivatives of the invention may be administered alone of with a pharmaceutically acceptable carrier, including acceptable adjuvants, vehicles, and excipients.

The effective dosage will depend on a variety of factors and it is well within the purview of a skilled physician to adjust the dosage for a given patient according to various parameters such as body weight, the goal of treatment, the highest tolerated dose, the specific formulation used, the route of administration and the like. Generally, dosage levels of between about 0.001 and about 100 mg/kg body weight per day of the antibody or derivative thereof are suitable. In some embodiments, the dose will be about 0.1 to about 50 mg/kg body weight per day of the antibody or derivative thereof. In other embodiments, the dose will be about 0.1 mg/kg body weight/day to about 20 mg/kg body weight/day. In still other embodiments, the dose will be about 0.1 mg/kg body weight/day to about 10 mg/kg body weight/day. Dosing may be as a bolus or an infusion. Dosages may be given once a day or multiple times in a day. Further, dosages may be given multiple times of a period of time. In some embodiments, the doses are given every 1-14 days. In some embodiments, the antibodies or derivatives thereof are given as a dose of about. 3 to 1 mg/kg i.p. In other embodiments, the antibodies of derivatives thereof are provided at about 5 to 12.5 mg/kg i.v. In still other embodiments, the antibodies or derivatives thereof are provided such that a plasma level of at least about 1 ug/ml is maintained.

Effective treatment may be assessed in a variety of ways. In one embodiment, effective treatment is determined by a slowed progression of tumor growth. In other embodiments, effective treatment is marked by shrinkage of the tumor (i.e., decrease in the size of the tumor). In other embodiments, effective treatment is marked by inhibition of metastasis of the tumor. In still other embodiments, effective therapy is measured by increased well-being of the patient including such signs as weight gain, regained strength, decreased pain, thriving, and subjective indications from the patient of better health.

The following Examples are provided to illustrate the present invention, and should not be construed as limiting thereof.

### EXAMPLES

### Example 1

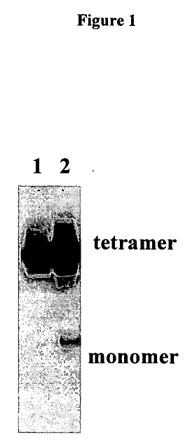

Binding of a monoclonal antibody to the tetrameric form of FR-α was shown by Western blot. Briefly, SK-Ov-3 and IGROV tumor cells were grown in nude mice and excised. Tumor tissues were lysed in RIPA buffer with 15-20 strokes in a 2 ml Dounce tissue homogenizer. Insoluble material was removed by centrifugation and the total protein of the supernate was determined using a BiORad protein Assay. In different experiments, either 5 ug or 20 ug of protein was run on a 4-12% Bis-Tris gel (MES) under non-reducing conditions. The electrophoresed protein was transferred to a PVDF membrane. The membrane was blocked in Blotto (5% milk, 0.05% TBS-T). A 1:100 dilution of culture supernate from LK26 hybridoma cells and total concentration of 0.1% NaN₃ was added directly to the Blotto blocking solution as the primary antibody, and the membrane was incubated overnight. The membrane was washed in 0.05% TBS-T and the secondary antibody (horseradish peroxidase labeled goat α-mouse IgG (heavy and light chains)) in Blotto blocking solution was added. The membrane was developed using Super Signal West Pico ECL reagent. The results are shown in FIG. 1 (lane 1, SK-Ov-3; lane 2, IGROV). The results indicate that certain tumors that overexpress FR-α, favor the production of tetrameric FR-α over monomeric FR-α. This finding can be exploited by monoclonal antibodies that specifically recognize the tetrameric form of FR-α for the destruction of tumor tissue, while leaving normal tissue (which generally expresses the monomeric form of FR-α) unscathed.

### Example 2

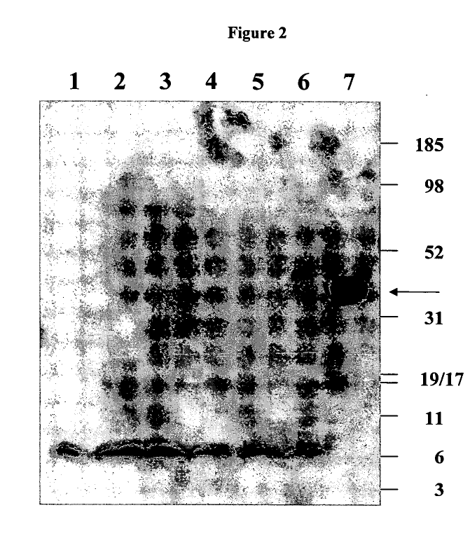

100721 Expression of FR-α was also assessed in Escherichia coli. Briefly, a plasmid containing the coding sequence for FR-α with a histidine tag (pBAD-His-hFR-α) was transfected into E. coli cells. A culture of E. coli containing plasmid pBAD-His-hFR-α was grown to OD₆₀₀=1.0. Thereafter, arabinose was added to a final concentration of 0.2%, and samples were taken at the time points indicated in FIG. 2. E. coli lysates were prepared by adding 25 ml of 4×LDS sample buffer to 65 ml culture. JAR cells were propagated in RPMI 1640 medium containing 10% FBS, L-glutamine, sodium pyruvate, non-essential amino acids, and penicillin/streptomycin. The medium was removed from the cells and RIPA buffer was added directly to the culture plates to lyse the cells for JAR cell extract controls. Samples were separated on a 4-12% NuPAGE gel (MES) and transferred to a PVDF membrane. After overnight blocking in TBST+5% milk, the membrane was probed with 1:1000 dilution of mAb LK26 for 1 hr followed by a 1:10000 dilution of secondary antibody (goat α-mouse Ig conjugated to horseradish peroxidase) for 1 hr. Detection of the antibody was performed with Pierce Super Signal femto after an exposure of 5 minutes. The results are shown in FIG. 2 (lane 1, E. coli+pBAD-His-hFRa, induced 180 min.; lane 2, E. coli+pBAD-His-hFRa, induced 90 min.; lane 3, E. coli+pBAD-His-hFRa, induced 60 min.; lane 4, E. coli+pBAD-His-hFRa, induced 30 min.; lane 5, E. coli+pBAD-His-hFRa, induced 15 min.; lane 6, E. coli+pBAD-His-hFRa, uninduced; lane 7, JAR cell extract). The results show that the E. coli cells produce only the monomeric form of FR-— a, and do not produce the tetrameric form of FR-α.

### Example 3

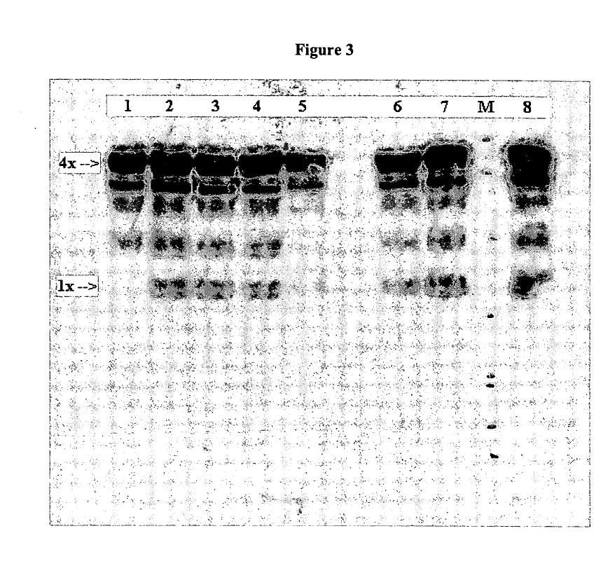

To demonstrate that the tetrameric FR-α was not an artifact of aggregation in Triton X-100 micelles as described by Holm et al. (1997) Biosci. Reports 17(4):415-427, extracts of tumors were diluted in either 1×RIPA (1% Triton X-100, 0.1% SDS, 180 mM NaCl, 20 mM potassium phosphate, pH=7.2) or 1×PBS (150 mM NaCl, 20 mM potassium phosphate, pH=7.2). For all samples, 1 ug/ul of stock IGROV extract was used. After dilution, 4×LDS sample buffer was added to each sample to a final concentration of lx. The samples were loaded on a 4-12% Bis-Tris gel in MES running buffer. Following electrophoresis, the protein was transferred to a PVDF membrane. The membrane containing the transferred protein was blocked for 48 hrs at room temperature in Blotto (5% skim milk, 1×TBS, 0.05% Tween-20). The membrane was developed by incubating the membrane with a primary antibody (1 ug/ml LK26 antibody) followed by washing, then incubation with a secondary antibody (HRP-conjugated goat α-mouse IgG in Blotto). Following another washing step, the membrane was developed using a Super Signal West Pico ECL reagent and exposed for 1 minute. The results are shown in FIG. 3 (lane 1, 1:100 dilution in PBS; lane 2, 1:50 dilution in PBS; lane 3, 1:25 dilution in PBS; lane 4, 1:10 dilution in PBS; lane 5, 1:100 dilution in RIPA; lane 6, 1:25 dilution in RIPA; lane 7, 1:10 dilution in RIPA; M, molecular weight markers, lane 8, 1:1 dilution in RIPA) Arrows indicate monomer (1×) and tetramer (4×). No treatment disrupted the tetrameric form of FR-α. The results indicate that certain tumors that over express FR-α express a tetrameric form of FR-α that has only been shown previously as artifacts of gel filtration sample preparations.

What is claimed:

1. An antibody that specifically binds to the tetrameric form of FR-α wherein said antibody is distinguished from mAb LK26 in that (a) said antibody binds to an epitope other than the epitope of mAb LK26; (b) said antibody binds with greater affinity than mAb LK26; or (c) said antibody out-competes mAb LK26 for binding to said tetrameric form of FR-α.

2. The antibody of claim 1 wherein the affinity of said antibody is at least about 1×10⁻⁷M.

3. The antibody of claim 1 wherein the affinity of said antibody is at least about 1×10⁻⁸M.

4. The antibody of claim 1 wherein the affinity of said antibody is at least about 1×10⁻⁹M.

5. The antibody of claim 1 wherein the affinity of said antibody is at least about 1×10⁻¹⁰M.

6. The antibody of claim 1 wherein said epitope is a disulfide-dependent epitope.

7. The antibody of claim 1 wherein said antibody is a chimeric antibody.

8. The antibody of claim 7 wherein said chimeric antibody is a human-mouse chimeric antibody.

9. The antibody of claim 1 wherein said antibody is a humanized antibody.

10. The antibody of claim 1 wherein said antibody is a fully human antibody.

11. A hybridoma cell that expresses the antibody of claim 1.

12. A polynucleotide encoding the antibody of claim 1.

13. A vector comprising the polynucleotide of claim 12.

14. An expression cell comprising the vector of claim 13.

15. A method of producing an antibody that specifically binds to the tetrameric form of FR-α wherein said antibody is distinguished from mAb LK26 in that (a) said antibody binds to an epitope other than the epitope of mAb LK26; (b) said antibody binds with greater affinity than mAb LK26; or (c) said antibody out-competes mAb LK26 for binding to said tetrameric form of FR-α, said method comprising the step of culturing the hybridoma of claim 11.

16. A method of producing an antibody that specifically binds to the tetrameric form of FR-α wherein said antibody is distinguished from mAb LK26 in that (a) said antibody binds to an epitope other than the epitope of mAb LK26; (b) said antibody binds with greater affinity than mAb LK26; or (c) said antibody out-competes mAb LK26 for binding to said tetrameric form of FR-α, said method comprising the step of culturing the expression cell of claim 14.

17. The expression cell of claim 14 wherein said cell is a mammalian cell.

18. The method of claim 16 wherein said expression cell is a mammalian cell.

19. A method of inhibiting the growth of dysplastic cells associated with increased expression of FR-α comprising administering to a patient with such dysplastic cells a composition comprising an antibody that specifically binds to the tetrameric form of FR-α wherein said antibody is distinguished from LK26 in that (a) said antibody binds to an epitope other than the epitope of LK26, (b) said antibody binds with greater affinity than LK26; or (c) said antibody out-competes mAb LK26 for binding to said tetrameric form of FR-α.

20. The method of claim 1 wherein said antibody is a monoclonal antibody.

21. The method of claim 1 wherein said dysplastic cells are ovarian carcinoma cells.

22. The method of claim 19 wherein said patient is a human patient.

23. The method of claim 19 wherein said antibody is conjugated to a chemotherapeutic agent.

24. The method of claim 23 wherein said chemotherapeutic agent is a radionuclide.

25. The method of claim 24 wherein said radionuclide is selected from the group consisting of Lead-212, Bismuth-212, Astatine-211, Iodine-131, Scandium-47, Rhenium-186, Rhenium-188, Yttrium-90, Iodine-123, Iodine-125, Bromine-77, Indium-111, Boron-10 and Actinide.

26. The method of claim 23 wherein said chemotherapeutic agent is selected from the group consisting of ricin, modified Pseudomonas enterotoxin A, calicheamicin, adriamycin, and 5-fluorouracil.

27. The antibody of claim 1 wherein antibody is conjugated to a chemotherapeutic agent.

28. The antibody of claim 27 wherein said chemotherapeutic agent is a radionuclide.

29. The antibody of claim 28 wherein said radionuclide is selected from the group consisting of Lead-212, Bismuth-212, Astatine-211, Iodine-131, Scandium-47, Rhenium-186, Rhenium-188, Yttrium-90, Iodine-123, Iodine-125, Bromine-77, Indium-111, Boron-10 and Actinide.

30. The antibody of claim 27 wherein said chemotherapeutic agent is selected from the group consisting of ricin, modified Pseudomonas enterotoxin A, calicheamicin, adriamycin, and 5-fluorouracil.
